(19) United States
(12) Patent Application Publication
(10) Pub. No.: US 2023/0263920 A1
(43) Pub. Date: Aug. 24, 2023

(54) KITCHEN STERILIZATION LAMP

(71) Applicant: Bestter (Xiamen)Technology Inc., Xiamen City (CN)

(72) Inventors: BinHua Gong, Xiamen City (CN); HaiFeng Li, Xiamen City (CN); Jun Jiang, Xiamen City (CN); GuoQiang Xu, Xiamen City (CN)

(21) Appl. No.: 17/744,367

(22) Filed: May 13, 2022

(30) Foreign Application Priority Data
Feb. 24, 2022 (CN) 202220406410.6

(51) Int. Cl. A61L 2/10

(52) U.S. Cl. CPC A61L 2/10; A61L 2202/11; A61L 2202/121; A61L 2202/14; A61L 2202/16

(57) ABSTRACT

A kitchen sterilization lamp is provided, which is used to be placed at the sewage outlet of the kitchen sink with a waste processor connected below, including a sterilization lamp holder, a sterilization lamp source and a power supply. The sterilization lamp is located on the sewage outlet of the kitchen sink, the sterilization lamp source is connected below the sterilization lamp holder and electrically connected to the power supply. The sterilization lamp source is in a rod shape, and the sterilization lamp holder extends vertically downward into the waste processor to irradiate the inner cavity of the waste processor. The present disclosure realizes the sterilization of the inner cavity of the waste processor and avoids the breeding of bacteria in the inner cavity of the waste processor.

### TECHNICAL FIELD

The present disclosure relates to the technical field of sterilization lamp, in particular to a kitchen sterilization lamp.

### BACKGROUND

The existing waste processor used in the kitchen sink is arranged below the sewage outlet of the kitchen sink. The kitchen waste discharged from the sewage outlet of the kitchen sink enters the inner cavity of the waste processor. The waste processor smashes the food residue and other kitchen waste and discharges it to the sewer pipe. Due to the large amount of water and organic matter in food waste, the inner cavity of the waste processor is highly susceptible to bacterial growth, resulting in a foul odor.

### SUMMARY

The object of the present disclosure is to provide a kitchen sterilization lamp to overcome the above defects and realize the sterilization of the inner cavity of the waste processor, so as to avoid the breeding of bacteria in the inner cavity of the waste processor.

In order to achieve the above object, a kitchen sterilization lamp is provided as a solution of the present disclosure, which is configured to be placed at a sewage outlet of a kitchen sink with a waste processor connected below, including a sterilization lamp holder, a sterilization lamp source and a power supply; the sterilization lamp holder is located on the sewage outlet of the kitchen sink, the sterilization lamp source is connected below the sterilization lamp holder and electrically connected to the power supply, and the sterilization lamp source is in a rod shape and extends vertically downward from the sterilization lamp holder into the waste processor to irradiate an inner cavity of the waste processor.

Further, the sterilization lamp source is detachably connected to the sterilization lamp holder.

Further, the kitchen sterilization lamp further includes a placement seat; the placement seat is configured to be placed outside the kitchen sink, the top of the placement seat is formed with a contact surface suitable for bearing the lower surface of the sterilization lamp seat, and the contact surface is provided with an insertion hole at a position corresponding to the sterilization lamp source for the insertion of the sterilization lamp source.

Further, a photosensitive switch is arranged in the sterilization lamp holder, the photosensitive switch is exposed at the lower surface of the sterilization lamp holder, and the photosensitive switch is electrically connected to the sterilization lamp source to control the sterilization lamp source to turn off when the photosensitive switch detects that a light intensity is greater than a preset value.

Further, a vibration sensor is arranged on the sterilization lamp holder, and the vibration sensor is electrically connected to the sterilization lamp source to control the sterilization lamp source to turn off when the sterilization lamp holder generates vibration.

Further, an upper part of the sterilization lamp holder is provided with a main switch, and the main switch is electrically connected to the sterilization lamp source to control the sterilization lamp source on or off.

Further, the sterilization lamp source is provided with a lamp source protective spring, and the lamp source protective spring spirally extends around the sterilization lamp source.

Further, the sterilization lamp source is provided with a lamp source protective cover, the lamp source protective cover is covered around the sterilization lamp source, and a light transmission hole is hollowed out on the lamp source protective cover.

Further, the power supply is arranged in the sterilization lamp holder, the surface of the sterilization lamp holder is provided with a charging port for the external connection with a power transmission device, and the charging port is electrically connected to the power supply to charge the power supply.

Further, the maximum cross section of the sterilization lamp holder is greater than the minimum cross section of the sewage outlet of the kitchen sink.

After adopting the above solution, the advantageous effect of the present disclosure is that the sterilization lamp holder is located on the sewage outlet of the kitchen sink, the sterilization lamp source is connected below the sterilization lamp holder and electrically connected to the power supply, the sterilization lamp source is in a rod shape and extends vertically downward from the sterilization lamp holder into the waste processor to irradiate the inner cavity of the waste processor. Then, after the kitchen waste is broken and discharged by the waste processor, the sterilization lamp holder is placed at the sewage outlet of the kitchen sink, so that the sterilization lamp source extends into the inner cavity of the waste processor, and the sterilization lamp source radiates ultraviolet rays to sterilize the inner cavity of the waste processor, so as to avoid the breeding of bacteria in the inner cavity of the waste processor, and there is no need for an appropriate connection structure on the waste processor, which has strong universality and can be applied to a variety of waste processors. After use, the sterilization lamp holder and sterilization lamp source can be taken out at any time to avoid damage to the sterilization lamp source when the waste processor works.

### BRIEF DESCRIPTION OF THE DRAWINGS

FIG. 1 is a partial exploded view showing the structure of the present disclosure;

FIG. 2 is a partial exploded view showing the structure in another direction of the present disclosure;

FIG. 3 is a partial cross-sectional view showing the structure of the present disclosure;

FIG. 4 is the structural view of the lamp source protective spring of the present disclosure;

FIG. 5 is a schematic diagram of the present disclosure in use state;

FIG. 6 is a three-dimensional structural diagram of the sterilization lamp holder of the present disclosure removed from the placement seat;

FIG. 7 is a cross-sectional view showing of the structure when the sterilization lamp holder of the present disclosure is placed on the placement seat;

FIG. 8 is the three-dimensional structural diagram of the drainage retaining ring of the present disclosure.

The reference numerals in the figure are: 1—waste processor; 2—sewage outlet of kitchen sink; 3—sterilization lamp holder, 4—sterilization lamp source, 5—power supply, 6—placement seat, 7—contact surface, 8—insertion hole, 9—photosensitive switch, 10—vibration sensor, 11—main switch, 12—lamp source protective spring, 13—charging port, 14—connection hole, 15—lamp source protective cover, 16—light transmission hole, 17—control panel, 18—first electrode, 19—second electrode, 20—dust cover, 21—shell, 22—upper cover, 23—elastic pad, 24—drainage retaining ring, 25—seal ring.

### DETAILED DESCRIPTION OF THE EMBODIMENTS

The present disclosure is described in detail below in combination with the attached drawings and specific embodiments.

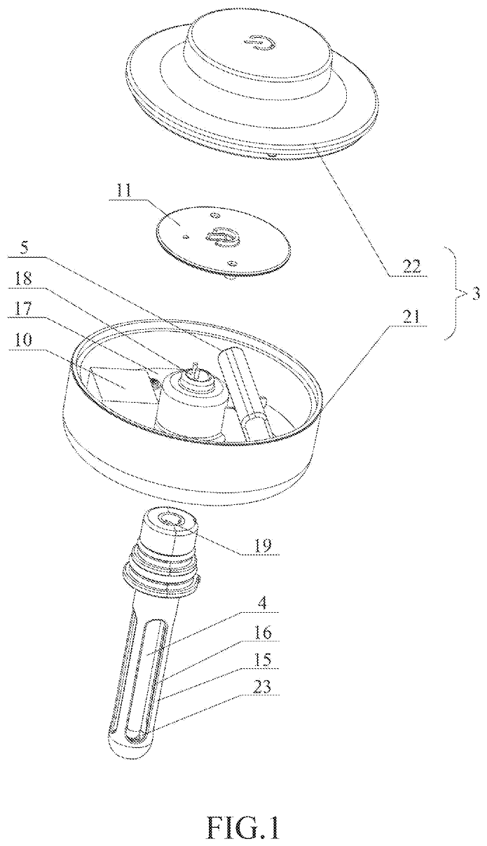

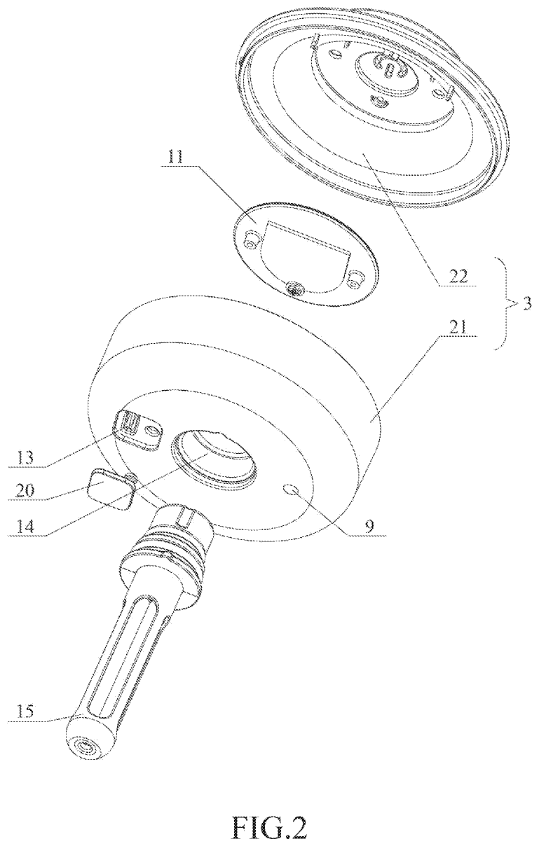

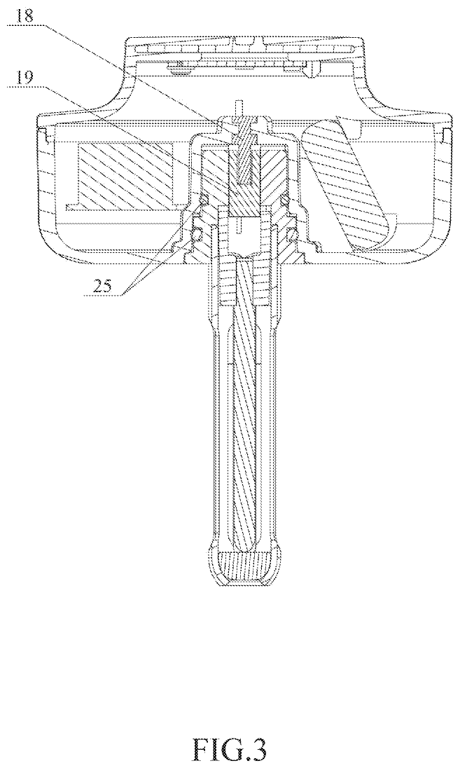

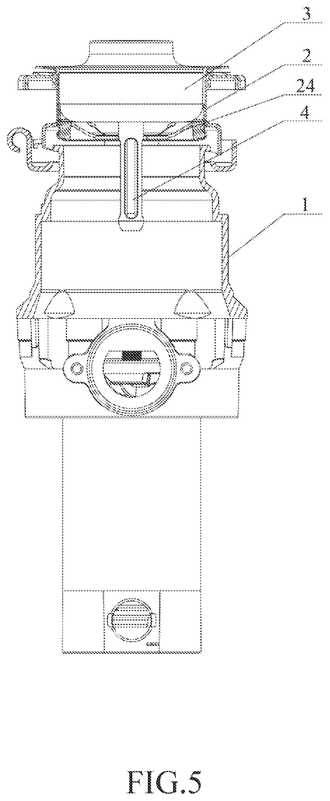

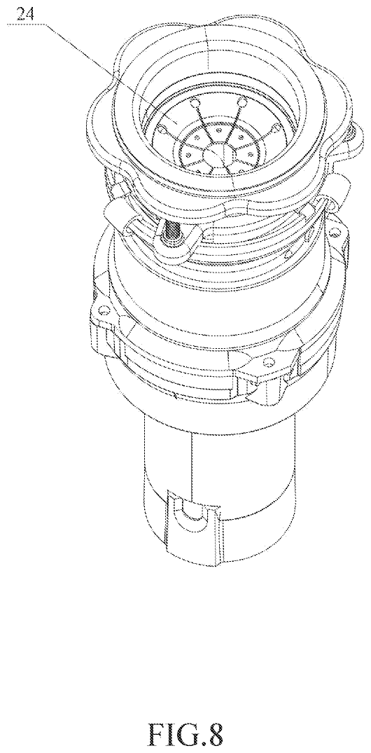

The present disclosure provides a kitchen sterilization lamp, as shown in FIGS. 1-8. Here, it is mainly combined with FIG. 1, FIG. 2 and FIG. 5, the kitchen sterilization lamp is used to place at the sewage outlet 2 of the kitchen sink connected to the waste processor 1 below, including a sterilization lamp holder 3, a sterilization lamp source 4 and a power supply 5. The maximum cross section of the sterilization lamp holder 3 is greater than the minimum cross section of the sewage outlet 2 of the kitchen sink, so as to locate on the sewage outlet 2 of the kitchen sink. In this embodiment, the sterilization lamp holder 3 is cylindrical, including a mutually buckled shell 21 and an upper cover 22. A cylindrical hole is formed at the top of the sewage outlet 2 of the kitchen sink, wherein the cross-section of the cylindrical hole is larger than the cross-section of the sterilization lamp holder 3. As shown in FIG. 8, the bottom of the sewage outlet 2 of the kitchen sink extends radially inward to form a drainage retaining ring 24. The sterilization lamp holder 3 is located on the drainage retaining ring 24, the sterilization lamp source 4 is connected below the sterilization lamp holder 3 and electrically connected to the power supply 5. In this embodiment, the sterilization lamp source 4 is detachably connected to the sterilization lamp holder 3 to avoid damage to the sterilization lamp source 4 during transportation, and it is convenient to replace the sterilization lamp source 4 when the sterilization lamp source 4 is damaged. Specifically, the detachable connection is plug-in connection. The bottom surface of the sterilization lamp holder 3 is formed with a connection hole 14 for inserting and fixing the top of the sterilization lamp source 4, and a seal ring 25 is arranged between the sterilization lamp source 4 and the sterilization lamp holder 3. There are two seal rings 25 in total, and the two seal rings 25 are sleeved on the outer circumference of the top of the sterilization lamp source 4 at intervals, so as to play the role of sealing, waterproof and fastening when the sterilization lamp source 4 is inserted into the connection hole 14. The power supply 5 is arranged in the sterilization lamp base 3, the control panel 17 is arranged in the sterilization lamp holder 3, and the power supply 5 is electrically connected to the control panel 17. Combined with FIG. 3, the first electrode 18 is arranged in the connection hole 14, and the control panel 17 is electrically connected to the first electrode 18 (not shown in the attached FIG.). The sterilization lamp source 4 is provided with a second electrode 19 matched with the first electrode 18 to realize the electrical connection between the sterilization lamp source 4 and the power supply 5 after the sterilization lamp source 4 is inserted into the connection hole 14. The sterilization lamp source 4 is in a rod shape, extending vertically downward from the sterilization lamp holder 3 into the waste processor 1, and the sterilization lamp source 4 is an ultraviolet lamp, which irradiates the inner cavity of the waste processor 1 with ultraviolet radiation.

Here, as shown in FIG. 1 and FIG. 2, the upper part of the sterilization lamp holder 3 is provided with a main switch 11. The main switch 11 is electrically connected to the control panel 17 and then electrically connected to the sterilization lamp source 4. In this embodiment, the main switch 11 is an inductive switch, and the finger clicks on the top of the main switch 11 to turn on or off the sterilization lamp source 4. The sterilization lamp holder 3 is provided with a photosensitive switch 9 and a vibration sensor 10. The photosensitive switch 9 is exposed at the lower surface of the sterilization lamp holder 3, the photosensitive switch 9 is electrically connected to the control panel 17 and then electrically connected to the sterilization lamp source 4, so as to control the sterilization lamp source 4 to turn off when the photosensitive switch 9 detects that the light intensity is greater than the preset value. When the sterilization lamp holder 3 is located on the sewage outlet 2 of the kitchen sink, the drainage retaining ring 24 blocks the photosensitive switch 9, to prevent the ultraviolet light emitted after the sterilization lamp source 4 is turned on from affecting the normal operation of the photosensitive switch 9. The vibration sensor 10 is electrically connected to the control panel 17, so as to electrically connected to the sterilization lamp source 4 to control the sterilization lamp source 4 to turn off when the sterilization lamp holder 3 generates vibrations.

The main switch 11, the photosensitive switch 9 and the vibration sensor 10 work together. First, the main switch 11 is clicked or pressed by finger, in this embodiment, the main switch 11 is pressed for three seconds to make the control panel 17 enter the sterilization mode. At this time, the photosensitive switch 9 and the vibration sensor 10 detect the light intensity and vibration respectively. The drainage retaining ring 24 blocks the photosensitive switch 9 when the sterilization lamp holder 3 is statically located at the sewage outlet 2 of the kitchen sink. The photosensitive switch 9 detects that the light intensity is lower than the preset value, and the vibration sensor 10 detects that there is no vibration generated by the sterilization lamp holder 3, after the state is delayed for a predetermined time, the control panel 17 controls the sterilization lamp source 4 to turn on and radiate ultraviolet rays. Otherwise, an indicator light arranged on the sterilization lamp holder 3 will indicate an abnormality, and the indicator light is electrically connected to the control panel 17. The indicator light is any kind of existing LED lamp, which is of conventional design and is not given in the attached drawings. If the conditions for the opening of the sterilization lamp source 4 are not met within a predetermined time, the control panel 17 enters the shutdown mode. When the sterilization lamp source 4 is turned on, the wrong operation picks up the sterilization lamp holder 3, at this time, when the light intensity detected by the photosensitive switch 9 is greater than the preset value, or when the vibration of the sterilization lamp holder 3 is detected by the vibration sensor 10, the control panel 17 controls the sterilization lamp source 4 to turn off, and prompts the abnormality through the indicator light, so as to realize double protection during the operation of the sterilization lamp source 4, avoiding the ultraviolet radiation from the sterilization lamp source 4 to cause harm to the user.

As shown in FIG. 2, the surface of the sterilization lamp holder 3 is provided with a charging port 13 for external connection of a power transmission device. The charging port 13 can be any existing charging port such as Type-C and Micro USB. The charging port 13 is electrically connected to the power supply 5 to charge the power supply 5. In this embodiment, the charging port 13 is located on the lower surface of the sterilization lamp holder 3 and covered by a dust cover 20. When not charging, cover the dust cover 20 to prevent the charging port 13 from entering water and dust.

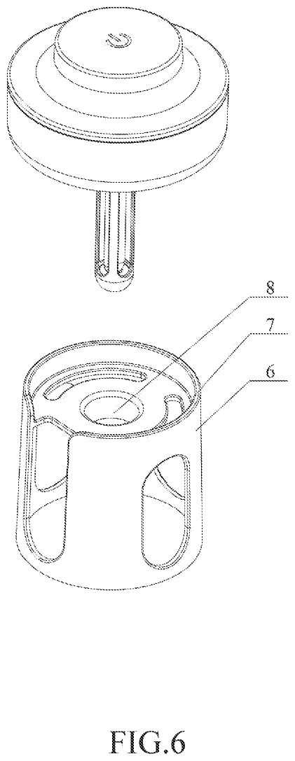

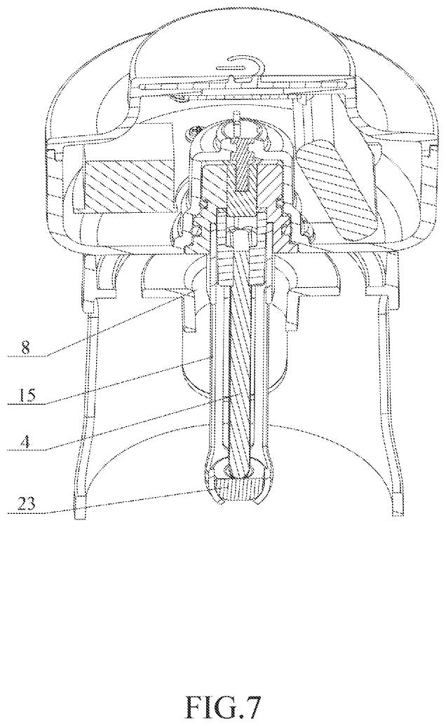

As shown in FIG. 6 and FIG. 7, preferably, it also includes a placement seat 6. The placement seat 6 is used to be placed on any table outside the kitchen sink. The top of the placement seat 6 is formed with a contact surface 7 suitable for bearing the lower surface of the sterilization lamp holder 3. The contact surface 7 is provided with an insertion hole 8 at the position corresponding to the sterilization lamp source 4 for the insertion of the sterilization lamp source 4. When the sterilization lamp is not used, the sterilizing lamp can be placed on the placement seat 6 to avoid damage to the sterilization lamp source 4 caused by accidental drop, collision, etc. The side wall of the placement seat 6 is hollowed out, which is convenient to keep the surface of the sterilization lamp dry, and can transmit light to the photosensitive switch 9 to prevent the sterilization lamp source 4 from being turned on incorrectly when placed on the placement seat 6.

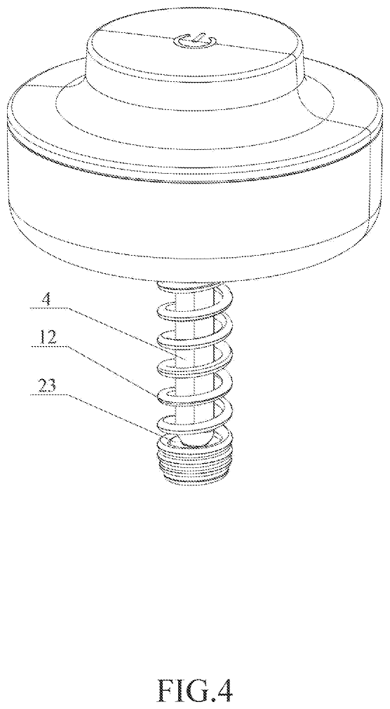

As shown in FIG. 1 and FIG. 4, the sterilization lamp source 4 is provided with a lamp source protective spring 12 or a lamp source protective cover 15. The lamp source protective spring 12 and the lamp source protective cover 15 are preferably made of steel, and the lamp source protective spring 12 spirally extends around the sterilization lamp source 4. The lamp source protective cover 15 is covered on the periphery of the sterilization lamp source 4, the lamp source protective cover 15 is hollowed out and provided with a light transmission hole 16. Elastic pads 23 are arranged between the lamp source protective spring 12, the lamp source protective cover 15 and the sterilization lamp source 4 to slow down the damage caused by vibration to the sterilization lamp source 4 and protect the sterilization lamp source 4, so as to realize the protection of the sterilization lamp source 4 and prevent mechanical damage, and it also ensures that the ultraviolet radiation emitted by the sterilization lamp source 4 is emitted as much as possible.

The description above is only a preferred embodiment of the present disclosure and is not a limitation on the design of the present disclosure. All equivalent changes made based on the design of the present disclosure fall into the scope of the present disclosure.

What is claimed is:

1. A kitchen sterilization lamp, which is configured to be placed at a sewage outlet (2) of a kitchen sink with a waste processor (1) connected below, comprising a sterilization lamp holder (3), a sterilization lamp source (4) and a power supply (5); the sterilization lamp holder (3) is located on the sewage outlet (2) of the kitchen sink, the sterilization lamp source (4) is connected below the sterilization lamp holder (3) and electrically connected to the power supply (5), and the sterilization lamp source (4) is in a rod shape and extends vertically downward from the sterilization lamp holder (3) into the waste processor (1) to irradiate an inner cavity of the waste processor (1).

2. The kitchen sterilization lamp according to claim 1, wherein the sterilization lamp source (4) is detachably connected to the sterilization lamp holder (3), and a seal ring (25) is arranged between the sterilization lamp source (4) and the sterilization lamp holder (3).

3. The kitchen sterilization lamp according to claim 1, wherein the kitchen sterilization lamp further comprises a placement seat (6); the placement seat (6) is configured to be placed outside the kitchen sink, a top of the placement seat (6) is formed with a contact surface (7) suitable for bearing a lower surface of the sterilization lamp seat (3), and the contact surface (7) is provided with an insertion hole (8) at a position corresponding to the sterilization lamp source (4) for an insertion of the sterilization lamp source (4).

4. The kitchen sterilization lamp according to claim 1, wherein a photosensitive switch (9) is arranged in the sterilization lamp holder (3), the photosensitive switch (9) is exposed at the lower surface of the sterilization lamp holder (3), and the photosensitive switch (9) is electrically connected to the sterilization lamp source (4) to control the sterilization lamp source (4) to turn off when the photosensitive switch (9) detects that a light intensity is greater than a preset value.

5. The kitchen sterilization lamp according to claim 4, wherein a bottom of the sewage outlet (2) of a kitchen sink extends radially inward to form a drainage retaining ring (24); and the drainage retaining ring (24) blocks the photosensitive switch (9) when the sterilization lamp holder (3) is located on the sewage outlet (2) of the kitchen sink.

6. The kitchen sterilization lamp according to claim 1, wherein a vibration sensor (10) is arranged on the sterilization lamp holder (3), and the vibration sensor (10) is electrically connected to the sterilization lamp source (4) to control the sterilization lamp source (4) to turn off when the sterilization lamp holder (3) generates vibration.

7. The kitchen sterilization lamp according to claim 1, wherein an upper part of the sterilization lamp holder (3) is provided with a main switch (11), and the main switch (11) is electrically connected to the sterilization lamp source (4) to control the sterilization lamp source (4) on or off.

8. The kitchen sterilization lamp according to claim 1, wherein the sterilization lamp source (4) is provided with a lamp source protective spring (12) or a lamp source protective cover (15), the lamp source protective spring (12) spirally extends around the sterilization lamp source (4), the lamp source protective cover (15) is covered around the sterilization lamp source (4), and a light transmission hole (16) is hollowed out on the lamp source protective cover (15).

9. The kitchen sterilization lamp according to claim 1, wherein the power supply (5) is arranged in the sterilization lamp holder (3), a surface of the sterilization lamp holder (3) is provided with a charging port (13) for an external connection with a power transmission device, and the charging port (13) is electrically connected to the power supply (5) to charge the power supply (5).

10. The kitchen sterilization lamp according to claim 1, wherein a maximum cross section of the sterilization lamp holder (3) is greater than a minimum cross section of the sewage outlet (2) of the kitchen sink.
